(19) United States
(12) Patent Application Publication
(10) Pub. No.: US 2024/0173108 A1
(43) Pub. Date: May 30, 2024

(54) METHOD OF PRODUCING A DENTAL RESTORATION

(71) Applicant: Ivoclar Vivadent AG, Schaan (LI)

(72) Inventors: Eser Atilim, Lindau (DE); Hendrik John, Buchs (CH)

(21) Appl. No.: 18/520,305

(22) Filed: Nov. 27, 2023

(30) Foreign Application Priority Data
Nov. 28, 2022 (EP) 22209976.4

(51) Int. Cl. A61C 13/00; B33Y 10/00; B33Y 30/00; B33Y 50/00; B33Y 80/00

(52) U.S. Cl. CPC A61C 13/0019; B33Y 10/00; B33Y 30/00; B33Y 50/00; B33Y 80/00

(57) ABSTRACT

A method of producing a dental restoration, including the steps of determining (S101) a first spatial region of the dental restoration that is subject to a higher load than a second spatial region of the dental restoration; and producing (S102) the dental restoration in the first spatial region with a different production material than in the second spatial region.

### CROSS-REFERENCE TO RELATED APPLICATIONS

This application claims priority to European Application No. 22209976.4 filed on Nov. 28, 2022, the disclosure of which is incorporated herein by reference in its entirety.

### TECHNICAL FIELD

The present invention relates to a method of producing a dental restoration and a production device for producing a dental restoration.

### BACKGROUND

In the digital production of crowns and bridges, general load cases are assumed for these crowns and bridges. This either restricts the number of bridge units or these parts are oversized in terms of wall thicknesses. This results in the mechanical properties of the materials used being many times higher than the loads that actually occur.

US 20070015110 and 20150320525 are directed to dental restorative devices and methods of making and are hereby incorporated by reference in their entirety.

### SUMMARY

It is the technical object of the present invention to improve the production of dental restorations.

This technical object is solved by subject-matter according to the independent claims. Technically advantageous embodiments are the subject of the dependent claims, the description and the drawings.

According to a first aspect, the technical object is solved by a method of producing a dental restoration, comprising the steps of determining a first spatial region of the dental restoration that is subject to a higher load than a second spatial region of the dental restoration; and producing the dental restoration in the first spatial region with a different production material than in the second spatial region. The method allows material properties to be optimally utilized and a range of indications to be expanded. Stress peaks within the dental restoration can be absorbed by adjusting the material accordingly. The dental restoration can therefore be produced with a lower material input.

In a technically advantageous embodiment of the method, the first spatial region is determined by the internal stress being above a predetermined value in this spatial region. This achieves the technical advantage, for example, that the first spatial region can be determined in a simple manner.

In another technically advantageous embodiment of the method, the load is calculated using a finite element method. This achieves the technical advantage, for example, that the load within the dental restoration can be calculated with a high degree of accuracy.

In another technically advantageous embodiment of the method, predetermined forces are applied to the dental restoration for the finite element method. For example, the calculations can be based on worst-case scenarios in which maximum expected forces act on the most unfavorable points of the dental restoration. This achieves the technical advantage, for example, that the dental restoration can be calculated with a high degree of strength (biaxial strength, fracture toughness). US 20100131244, 11787120, 20230218373, 20190110871 and 20220143922 are directed to systems and methods of using finite element methods and are hereby incorporated by reference in their entirety.

In a further technically advantageous embodiment of the method, the production material for the first and/or second spatial region is selected on the basis of a calculated load. This achieves the technical advantage, for example, that different strengths are achieved for the spatial regions and adapted to the load.

In a further technically advantageous embodiment of the method, the production material is doped differently in the first spatial region than in the second spatial region. This achieves the technical advantage, for example, that a sintering process can be carried out without sintering distortion and the production material has different strength values. In addition to the sintering properties, the mechanical properties, the optical properties and the aging resistance can also be adjusted by means of the doping.

As suitable strength values (flexural strength+fracture toughness) for differently yttrium-doped zirconium dioxide materials, the following values are common:

| Material | 3Y-TZP | 4Y-TZP | 5Y-TZP |
|---|---|---|---|
| Biaxial flexural | 1000 ± 200(*) | 750 ± 100(*) | 600 ± 50(*) |
| strength [MPa] | | | |
| Fracture Toughness | 5.00 ± 0.25 | 3.75 ± 0.25 | 2.40 ± 0.25 |
| [MPa√m] | | | |

In a further technically advantageous embodiment of the method, in the first spatial region a production material with a higher strength than in the second spatial region is used. This achieves the technical advantage, for example, that a wall thickness can be reduced in these regions.

In a further technically advantageous embodiment of the method, the dental restoration is produced by means of a three-dimensional printing process. This achieves the technical advantage, for example, that the production of the dental restoration can be carried out efficiently.

In a further technically advantageous embodiment of the method, the three-dimensional printing process uses a free-jet material deposition. This achieves the technical advantage, for example, that the production materials can be arranged in the respective spatial regions in a simple manner.

According to a second aspect, the technical object is solved by a production device for producing a dental restoration, comprising a determination device for determining a first spatial region of the dental restoration which is subjected to a higher load than a second spatial region of the dental restoration; and a production apparatus for producing the dental restoration in the first spatial region with a different production material than in the second spatial region. The production device achieves the same technical advantages as the method according to the first aspect.

In a technically advantageous embodiment of the production device, the determination device is configured to determine the first spatial region by the internal stress in this spatial region being above a predetermined value. This also achieves the technical advantage, for example, that the first spatial region can be determined in a simple manner.

In a further technically advantageous embodiment of the production device, the determination device is configured to calculate the load using a finite element method. This also achieves the technical advantage, for example, that the load can be calculated with a high degree of accuracy.

In a further technically advantageous embodiment of the production device, the production apparatus is configured to select the production material for the first and/or second spatial region on the basis of a calculated load. This also achieves the technical advantage, for example, of simplifying the production of the dental restoration.

In a technically advantageous embodiment of the production device, the production apparatus is configured to dope the production material in the first spatial region differently than in the second spatial region. This also achieves the technical advantage, for example, that a sintering process can be carried out without sintering distortion.

In a technically advantageous embodiment of the production device, the production apparatus comprises a 3D printer. This also achieves the technical advantage, for example, that the production of the dental restoration can be carried out efficiently.

The production device and/or determining device may include computer (s)/devices and server computer (s) to provide processing, storage, and input/output devices executing application programs and the like. The computer (s)/devices can also be linked through communications network to other computing devices. The communications network can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

I/O device interfaces for connecting various input and output devices include, but are not limited to e.g., keyboard, mouse, displays, printers, speakers, etc. A memory provides volatile storage for computer software instructions and data used to implement an embodiment of the present invention. Disk storage provides non-volatile storage for computer software instructions and data used to implement an embodiment of the present invention. A central processor unit can be used to provide for the execution of computer instructions.

In one embodiment, the processor routines and data are a computer program product, including a non-transitory computer-readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network (s)). Such carrier medium or signals may be employed to provide at least a portion of the software instructions for the present invention routines/program.

### BRIEF DESCRIPTION OF THE DRAWINGS

Exemplary embodiments of the invention are shown in the drawings and are described in more detail below, in which:

FIG. 1 shows a schematic view of the production of a dental restoration;

FIG. 2 shows a schematic view of a production device for producing a dental restoration; and

FIG. 3 shows a block diagram of a method for producing the dental restoration.

### DETAILED DESCRIPTION

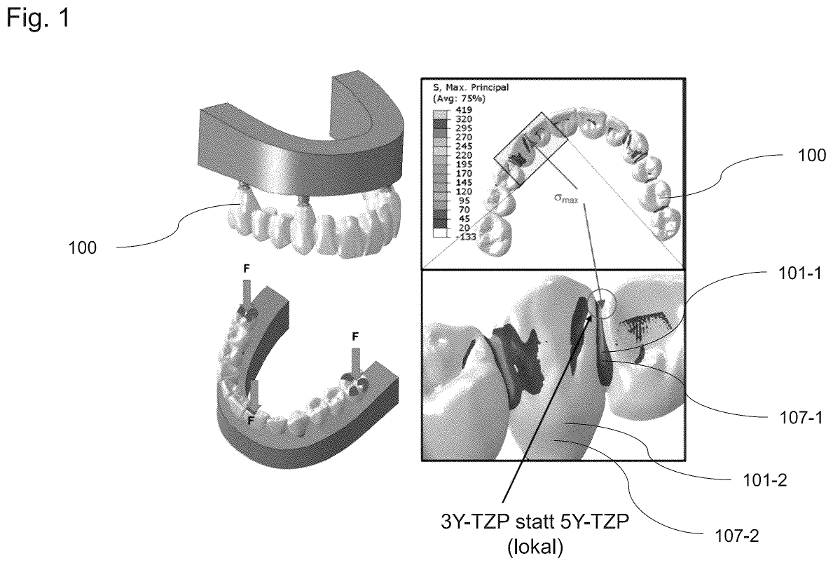

FIG. 1 shows a schematic view of the production of a dental restoration 100. The dental restoration 100 is, for example, a crown, a bridge, a partial prosthesis or a full prosthesis.

In order to better utilize the mechanical properties of the production materials 107-1 and 107-2, predefined load cases for the dental restoration 100 are first simulated using FEM software (Finite Element Method software). Through the FEM software, internal force histories and stresses can be calculated based on a three-dimensional model of the dental restoration 100 to determine weak points in the dental restoration 100. For easier identification of the underloaded or overloaded spatial regions (subvolumes), these can be marked by specific coloring. For example, spatial regions 101-1 within the dental restoration 100 in which an internal stress is above a predetermined value can be determined in this way.

For this purpose, the dental restoration 100 can be specifically loaded with forces that may occur during the use of the dental restoration 100. These forces act on the weakest points of the dental restoration 100. For example, if the dental restoration 100 is a bridge, the maximum force that can occur is applied to the center of the bridge. The FEM software then calculates how the stresses are distributed within the bridge. In this way, spatial regions 101-1 can be determined in which the stresses are particularly high.

However, real forces can also be determined by means of a pressure mat based on a real bite situation. The pressure mat measures the forces that occur when the teeth bite together. The forces determined can then be used by the FEM software for the dental restoration 100 to calculate the internal distribution of stress within the dental restoration 100 under real conditions.

The FEM software is also able to make recommendations for material adaptation because it knows the mechanical properties of the various available production materials 107-1 and 107-2. By knowing the underdetermined and/or overdetermined spatial regions 101-1 and 101-2 through the FEM analysis in the dental restoration 100, appropriate corrective measures can be taken. As a result, the probability of component failure can be minimized and a load guarantee can be provided to the customer. Furthermore, the software enables a plausibility check of the production materials 107-1 and 107-2 used.

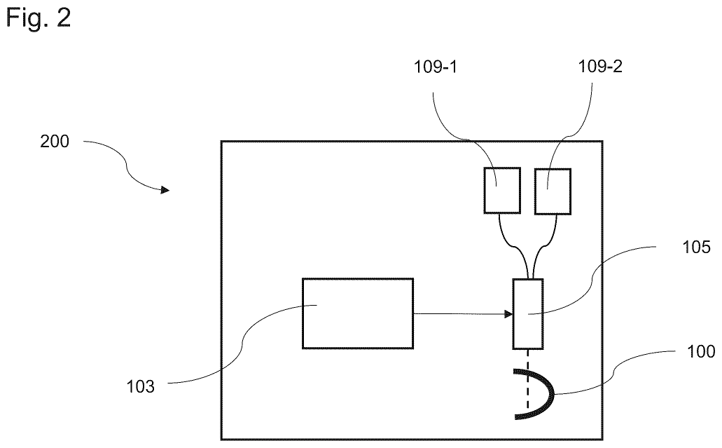

FIG. 2 shows a schematic view of a for producing the dental restoration 100. The production system 200 comprises a determination device 103 for determining a first spatial region 101-1 of the dental restoration 100 that is subjected to a higher load than a second spatial region 101-2 of the dental restoration 100.

The determination device 103 comprises, for example, a processor and a memory on which FEM software is executed. The determination device 103 is formed by a computer, for example.

The FEM software running on the determination device 103 uses a digital model of the dental restoration 100 to calculate those spatial regions in which higher mechanical loads occur than in other spatial regions. In this way, the digital model of the dental restoration 100 can be divided into different spatial regions 101-1 or 101-2. In this process, three or more spatial regions with different mechanical loads can also be defined within the dental restoration 100. A separate production material can be used for each of these spatial regions.

Subsequently, the determination device 103 sends control data to a production apparatus 105 with which the dental restoration is produced. The determination device 103 can automatically assign the respective production materials 107-1 and 107-2 to be used to different spatial regions 101-1 and 101-2 depending on the calculated loads. This can be performed because the determination device 103 knows the properties of the available production materials.

The production apparatus 105 uses a different production material 107-1 in the first spatial region 101-1 than in the second spatial region 101-2. This production material 107-1 may have a higher strength than the other production material 107-2.

For example, the production apparatus 105 uses an additive manufacturing process that selectively deposits a raw material in the form of droplets in a layer-by-layer manner, such as poly-jet modeling and multi-jet modeling. The production apparatus 105 comprises, for example, a first container 109-1 for the first production material 107-1 and a second container 109-2 for the second production material 107-2. The control signals from the determination device cause the production apparatus 105 to automatically use the assigned production material 107-1 or 107-2 when printing the respective spatial region 101-1 and 101-2.

The production system 200 may reduce the isotropy of the load distribution within the dental restoration 100. A different, such as stronger, production material 107-1 can be used for a more stressed or loaded spatial region 101-1, such as zirconium oxide with 3 weight percent yttrium oxide (3YTZP) instead of zirconium oxide with 5 weight percent yttrium oxide (5YTZP).

If the same production material 107 were used for both spatial regions 101-1 and 101-2, this would require thickening of the dental restoration 100, which is often undesirable for esthetic and haptic reasons. The production system 200 allows for a reduction in wall thickness for underloaded subvolumes for improved aesthetics.

This selective adjustment of material assignment can be performed as part of a multi-material additive manufacturing process in which selective material deposition occurs freely in three-dimensional space, such as in a free-jet material deposition in poly- or multi-jet modeling.

For example, in 3D inkjet printing, production materials 107-1 and 107-2 having a viscosity greater than 15 mPas, preferably greater than 150 mPas, and highly preferably greater than 200 mPas can be jetted at a suitable processing temperature. The selective material deposition of the material jetting can be used to build up smallest spatial regions 101-1 up to a size of a single voxel with different production materials 107-1 and 107-2.

If a user either changes the design of the dental restoration 100 or assigns other production materials 107-1 and 107-2 with different mechanical properties, this change can be made visible to the user. If the design and the production materials 107-1 and 107-2 are in order, the user can start the print job for the dental restoration 100. This is then automatically built up with the respective production materials 107-1 and 107-2 in the respective spatial regions 101-1 and 101-2.

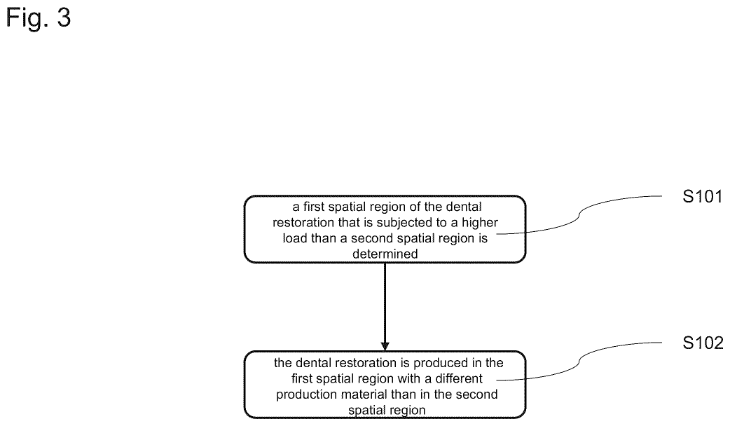

FIG. 3 shows a block diagram of a method for producing the dental restoration 100. In step S101, a first spatial region 101-1 of the dental restoration 100 is determined that is subjected to a higher load than a second spatial region 101-2 of the dental restoration 100. In step S102, the dental restoration 100 is produced in the first spatial region 101-1 with a different production material 107-1 than in the second spatial region 101-2. The method can be applied to all spatial regions 101-1 and 101-2 of the dental restoration 100.

During the processing of zirconium oxide slurries, the sintering properties can be locally changed in a spatial region by selectively doping the material layer differently in the green body stage. This leads to different strength regions in the final dental restoration 100 in the sintering process when different production materials 107-1 and 107-2 are used. Selective doping is performed, for example, by jetting an infiltration fluid into a previously applied and dried layer of material. In addition to the sintering properties, the mechanical properties, optical properties and aging resistance can also be adjusted by means of doping.

The method can identify spatial regions 101 in the dental restoration 100 that are either overdetermined or should be reinforced. Accordingly, other production materials 107-1 and 107-2 with different mechanical properties can be selectively and locally assigned. As a result, crowns or bridges made of final production material 107-1 and 107-2 (composite or ceramic), can be adapted to the individual load cases.

The scope of indications can be expanded and the load that occurs can be quantified and made visible. The dental technician can be supported when modeling the dental restoration by indicating spatial regions 101 where wall thicknesses can be reduced or should be increased. By assigning different production materials 107-1 and 107-2 with different mechanical properties to the respective spatial regions 101, load peaks can be absorbed without any additional increase in wall thickness.

All of the features explained and shown in connection with individual embodiments of the invention may be provided in different combinations in the subject matter of the invention to simultaneously realize their beneficial effects.

All method steps can be implemented by devices which are suitable for executing the respective method step. All functions that are executed by the features of the subject matter can be a method step of a method.

The scope of protection of the present invention is given by the claims and is not limited by the features explained in the description or shown in the figures.

### REFERENCE LIST

100 Dental restoration
101 Spatial region
103 Determination device
105 Production device
107 Production material
109 Container
200 Production system

1. A method of producing a dental restoration, comprising the steps of:
determining a first spatial region of the dental restoration that is subject to a higher load than a second spatial region of the dental restoration; and
producing the dental restoration in the first spatial region with a different production material than in the second spatial region.

2. The method according to claim 1,
wherein the first spatial region is determined by the internal strain in the first spatial region being above a predetermined value.

3. The method according to claim 2,
wherein the load is calculated using a finite element method.

4. The method according to claim 3,
wherein predetermined forces are applied to the dental restoration for the finite element method.

5. The method according to claim 1,
wherein the production material for the first and/or second spatial region is selected based on a calculated load.

6. The method according to claim 1,
wherein the production material is doped differently in the first spatial region than in the second spatial region.

7. The method according to claim 1,
wherein in the first spatial region a production material having a higher strength than in the second spatial region is used.

8. The method according to claim 1,
wherein the production of the dental restoration is performed by means of a three-dimensional printing process.

9. The method according to claim 8,
wherein the three-dimensional printing process uses free-jet material deposition.

10. A production device for producing a dental restoration, comprising:
a determination device for determining a first spatial region of the dental restoration which is subjected to a higher load than a second spatial region of the dental restoration; and
a production apparatus for producing the dental restoration in the first spatial region with a different production material than in the second spatial region.

11. The production device according to claim 10,
wherein the determination device is configured to determine the first spatial region by the internal stress being above a predetermined value in the first spatial region.

12. The production device according to claim 11,
wherein the determination device is configured to calculate the load using a finite element method.

13. The production device according to claim 10,
wherein the production apparatus is configured to select the production material for the first and/or second spatial region based on a calculated load.

14. The production device according to claim 10,
wherein the production apparatus is configured to dope the production material in the first spatial region differently than in the second spatial region.

15. The production device according to claim 10,
wherein the production apparatus comprises a 3D printer.
